(19) United States
(12) Patent Application Publication
(10) Pub. No.: US 2026/0083843 A1
(43) Pub. Date: Mar. 26, 2026

(54) IMMUNE CELLS TARGETING EPCAM AND MEDICAL USE THEREOF

(71) Applicant: SUZHOU IMMUNOFOCO BIOTECHNOLOGY CO., LTD, Suzhou, Jiangsu (CN)

(72) Inventor: Suqiong Wang, Suzhou (CN)

(73) Assignee: SUZHOU IMMUNOHEAD BIOTECHNOLOGY CO., LTD, Suzhou, JS (CN)

(21) Appl. No.: 18/869,768

(22) PCT Filed: Jun. 5, 2023
(86) PCT No.: PCT/CN2023/098386

§ 371 Date: Nov. 26, 2024

(30) Foreign Application Priority Data
Jun. 10, 2022 (CN) 202210656644.0

(51) Int. Cl. A61K 40/11; A61K 35/17; A61K 40/15; A61K 40/31; A61K 40/42; A61P 35/04; C07K 16/30

(52) U.S. Cl. CPC A61K 40/11; A61K 35/17; A61K 40/15; A61K 40/31; A61K 40/4254; A61P 35/04; C07K 16/30; A61K 2239/54; A61K 2239/59; C07K 2317/565; C07K 2317/622

(57) ABSTRACT

The present invention relates to the technical field of biomedicine, and specifically relates to immune cells targeting EpCAM and a medical use thereof. Provided is a use of immune cells in the preparation of a drug for preventing or treating a tumor; the immune cells are cells for adoptive immune cell therapy, which can target and kill EpCAM-expressing cells; and the tumor expresses EpCAM and is a metastatic tumor.

### CROSS-REFERENCE TO RELATED APPLICATIONS

This disclosure claims priority to Chinese Patent Application No. 202210656644.0 filed on Jun. 10, 2022, which is incorporated herein by reference in its entirety.

### TECHNICAL FIELD

The present disclosure relates to the technical field of biopharmaceuticals, and in particular, to an EpCAM-targeted immune cell and pharmaceutical use thereof.

### BACKGROUND

In recent years, the development of immunotherapy has brought profound changes to the field of cancer therapies, especially the immune checkpoint therapies represented by PD1/PDL1 pathway inhibitors and adoptive cell therapies represented by CAR-T. The adoptive cell therapies include TIL, NK, TCR-T, CAR-T, etc. Among them, the CAR-T therapy targeting CD19 has exhibited excellent clinical efficacy in B-cell tumors. So far, five CAR-T products have been approved in the US by the FDA for the treatment of B-cell leukemia or lymphoma, and two CAR-T products have been approved for marketing in China.

The concept of chimeric antigen receptors (CARs) was first proposed by scientists Zelig Eshhar, et al. CAR-T can highly specifically target tumor antigens without relying on the major histocompatibility complex (MHC). The CAR structure is composed of an extracellular single-chain variable fragment (scFv), a transmembrane region, a co-stimulatory region, and an intracellular signaling region. The first-generation CARs comprise only one signaling unit, mainly derived from the CD3(or FcRy subunit. The second-generation CARs, where a co-stimulatory factor is introduced on the basis of the first-generation CARs, have a co-stimulatory signaling domain and a signaling domain. In the third-generation CAR-T cells, two co-stimulatory factors such as CD28 and 4-1BB are introduced, which can enhance the anti-tumor effects of the T cells. On the basis of the existing antibody-recognition region and signaling region, in the fourth-generation CARs (TRUCK T cells), one or more constitutive or inducible expression components are added to enable the CAR-T cells to express specific proteins, thus enhancing the viability of CAR-T cells, promoting the infiltration of T cells into tumor tissues, and resisting the tumor inhibitory microenvironment.

CAR-T therapies, represented by CD19 CAR-T targeting a CD19 antigen, have exhibited excellent efficacy in hematological tumors. CD19 CAR-T has been approved in the treatment of adult B-cell type acute lymphoma (ALL), chronic lymphoma (CLL), non-Hodgkin's lymphoma (NHL), and other diseases, and most of the products have a complete response rate of above 70% in clinical applications.

Very good efficacy has not been achieved yet in the treatment of solid tumors. With the progress in the field of tumor therapies, more and more studies have found that the recurrence and metastasis of cancer are the main causes of death of cancer patients, and therefore, the control of the recurrence and metastasis of cancer is the key to prolong the life of cancer patients.

Epithelial cell adhesion molecule (EpCAM) is a major surface antigen associated with human cancer cells such as colon cancer, gastric cancer, breast cancer, ovarian cancer, and pancreatic cancer, and can be highly expressed in various human epithelial tissues, cancers, progenitor cells, and stem cells, such that the epithelial cell adhesion molecule has potential to become an important target of antitumor immunotherapy.

### SUMMARY

The present disclosure relates to use of an immune cell in the preparation of a medicament for preventing or treating a tumor, wherein:
the immune cell is a cell for an adoptive immune cell therapy, which can target and kill an EpCAM-expressing cell;
the tumor expresses EpCAM and is a tumor with a metastasis.

The present disclosure also relates to a method for preventing or treating a tumor in a patient in need thereof, which comprises administering to the patient a therapeutically effective amount of the immune cell as described above or a pharmaceutical composition.

It is found in the present disclosure for the first time that an immune cell capable of targeting and killing an EpCAM-expressing cell can effectively treat a tumor with metastasis, and use of a specific CAR-T cell in the tumor is verified.

### BRIEF DESCRIPTION OF THE DRAWINGS

In order to more clearly illustrate the specific embodiments of the present disclosure or technical solutions in the prior art, the drawings used for the specific embodiments and the prior art are briefly introduced below. It is obvious that the drawings in the following description are only illustrative of some embodiments of the present disclosure, while other drawings can be obtained by those of ordinary skill in the art according to these drawings without creative efforts.

FIG. 1 shows the fluorescence intensity of metastases in mice after treatment with EpCAM-CAR-T or a control in a CTC metastatic tumor model;

FIG. 2 shows the fluorescence intensity of metastases in the lungs and liver of mice after treatment with EpCAM-CAR-T or a control in a CTC metastatic tumor model;

FIG. 3 shows the fluorescence intensity of metastases in an abdominal metastasis model of gastric cancer after treatment with EpCAM-CAR-T or controls.

### DETAILED DESCRIPTION

References to embodiments of the present disclosure are provided in detail by means of one or more examples below. The examples are provided for illustrating rather than limiting the present disclosure. Actually, it is obvious to those skilled in the art that many modifications and variations can be made to the present disclosure without departing from the scope or spirit of the present disclosure. For example, a feature stated or described as part of one embodiment may be used in another embodiment to produce a further embodiment.

Unless otherwise stated, all terms (including technical and scientific terms) used to disclose the present disclosure have the same meaning as would normally be understood by those of ordinary skill in the art. With further guidance, subsequent definitions are used to better understand the teachings of the present disclosure. The terms used in the specification of the present disclosure are for the purpose of describing the specific examples only and are not intended to limit the present disclosure.

The term “and/or” or “or/and” as used herein includes any one of two or more relevant listed items, and any and all combinations of relevant listed items. Said any and all combinations include a combination of any two or more or all of the relevant listed items. It should be noted that in the present application, when at least three items are connected by at least two combinations of conjunctions selected from “and/or” and “or/and”, the technical solution undoubtedly encompasses technical solutions with items connected by “logical and” and technical solutions with items connected by “logical or”. For example, “A and/or B” includes three parallel solutions, i.e., A, B, and A+B. For another example, a technical solution of “A, and/or, B, and/or, C, and/or, D” includes any one of A, B, C, and D (i.e., a technical solution with items connected by “logical or”), and also includes any and all of the combinations of A, B, C, and D, i.e., combinations of any two or three of A, B, C, and D, and a combination of four of A, B, C, and D (i.e., a technical solution with items connected by “logical and”).

The terms “contain”, “comprise”, and “include” as used herein are synonyms, and are inclusive or open-ended but not exclusive of additional or uncited members, elements, or procedures.

A numeral range represented by endpoints as used herein includes all values and fractions within the range, as well as the endpoints.

A concentration value, as used herein, includes fluctuations within a certain range. For example, it may fluctuate within a corresponding precision range. For example, for 2%, a fluctuation within the range of ±0.1% may be permitted. For greater values or those that require no fine controls, larger fluctuations may also be permitted. For example, for 100 mM, fluctuations within the range of ±1%, ±2%, ±5%, etc., may be permitted.

In the present disclosure, the description related to “multiple”, unless otherwise defined, means a quantity greater than or equal to 2.

In the present disclosure, the technical features described in an open-ended manner include a closed-ended embodiment consisting of the enumerated features and an open-ended embodiment comprising the enumerated features.

In the present disclosure, the term “preferred”, “preferable”, or “preferably” only describes embodiments or examples with better results and should not be construed as limitations to the protection scope of the present disclosure. In the present disclosure, the term “optional” or “optionally” refers to non-essential, i.e., being selected from either of two parallel solutions of “presence” or “absence”. If there are multiple “optional” in one embodiment, the “optional” are each independent unless otherwise specified or contradicted or constrained by each other.

A first aspect of the present disclosure relates to use of an immune cell in the preparation of a medicament for preventing or treating a tumor, wherein
the immune cell is a cell for an adoptive immune cell therapy, which can target and kill an EpCAM-expressing cell;
the tumor expresses EpCAM and is a tumor with a metastasis.

In some embodiments, the tumor is a gastrointestinal tumor, pancreatic cancer, ovarian cancer, or bladder cancer.

In some embodiments, the gastrointestinal tumor is gastric cancer or colorectal cancer.

In some embodiments, the tumor is gastric cancer with a peritoneal metastasis.

In some embodiments, the tumor is colorectal cancer with a liver metastasis.

In some embodiments, the tumor is an advanced tumor.

In some embodiments, the tumor metastasizes through circulating tumor cells.

In the present disclosure, the adoptive immune cell therapy includes an NK therapy, a LAK therapy, a DC therapy, a CIK therapy, a TIL therapy, a DC-CIK therapy, a CAR-T therapy, a TCR-T therapy, a CAR-NK therapy, or a TCR-NK therapy.

In a preferred embodiment, the immune cell is a T cell, an NK cell, or a DC cell.

In some embodiments, the T cell is any one of a helper T cell, a cytotoxic T cell, a memory T cell, a regulatory T cell, a MAIT cell, or a T6T cell.

In some embodiments, the immune cell is a TCR-T cell, a CAR-T cell, or a CAR-NK cell.

In some embodiments, the immune cell is an autoimmune cell.

In some other embodiments, the immune cell is an allogeneic immune cell.

In some preferred embodiments, the immune cell expresses a chimeric antigen receptor, and the chimeric antigen receptor has an extracellular antigen-recognition domain for recognizing an EpCAM antigen.

As used herein, the “chimeric antigen receptor (CAR)” refers to a fusion protein comprising an extracellular domain capable of binding to an antigen, a transmembrane domain derived from a polypeptide different from the polypeptide where the extracellular domain is derived, and at least one intracellular domain. The “chimeric antigen receptor (CAR)” is sometimes referred to as “chimeric receptor” or “chimeric immune receptor (CR)”. The term “extracellular domain capable of binding to an antigen” refers to any oligopeptide or polypeptide capable of binding to a specific antigen. The “intracellular domain” refers to any oligopeptide or polypeptide known to function in cells as a domain that transmits signals to activate or inhibit a biological process.

In some embodiments, the chimeric antigen receptor comprises a hinge region, a transmembrane region, and an intracellular signaling region.

As used herein, the “region” or “domain” in the chimeric antigen receptor refers to a region of a polypeptide that can be folded into a specific structure independently of other regions. Such “regions” or “domains” may be sequences derived from murine or other animals, preferably a human sequence. In addition, the “region” or “domain” is understood to mean a known sequence, which may be a full-length or partially active segment, when not specifically distinguished or emphasized.

In some embodiments, the hinge region is selected from a hinge region of CD8, CD28, IgG1, IgG4, 4-1BB, ICOS, OX40, CD40, CD80, or CD7, or a CH3 or CH2-CH3 constant region.

In some embodiments, the transmembrane region is selected from a transmembrane region of CD8a, CD28, CD4, ICOS, CD7, CD2, CD80, CD40, OX40, CD27, LFA-1, 4-1BB, ICOS, CD3(, or CD3R.

In some embodiments, the intracellular signaling region comprises a CD3(signaling domain. In some embodiments, the intracellular signaling region further comprises a protein or an intracellular signaling region (or co-stimulatory region) thereof selected from those shown in the table below; in some embodiments, the intracellular signaling region further comprises one or more selected from the following proteins or intracellular signaling regions thereof. CD28, 4-1BB, OX40, ICOS, CD27, MYD88, KTR2DS2, DAP10, DAP12, CD3(, TLRs, CD2, LFA-1, CD8a, CD40, CD80, and CD3g.

In some embodiments, the antigen-recognition domain is a single-chain antibody (preferably scFv).

The single-chain antibody may be a chimeric, humanized, or human antibody fragment capable of recognizing an EpCAM antigen-binding domain.

In the present disclosure, the term “humanization” or “humanization treatment” refers to a replacement of an animal-derived (e.g., murine) antibody sequence with a human antibody sequence, thereby reducing or eliminating a human anti-mouse antibody (HAMA) response. Such a replacement may be a framework replacement, for example, a replacement of the FR sequences in the variable region with those of human origin, and/or a replacement of the constant region of the antibody (if present) to that of human origin. Such a replacement may also be a conversion of a murine monoclonal antibody to a human antibody (i.e., CDRs are also replaced) by chain shuffling by available means such as phage antibody library technology. It should be noted that in the humanization process, the replaced human sequence may comprise partial amino acid replacements or additions or deletions such that the substituted sequence may not be an exact copy of the expressed human immunoglobulin sequence or germline gene sequence. The antibodies so produced may be referred to as human-mouse chimeric antibodies, humanized antibodies, or human antibodies.

In some embodiments, the sc-Fv comprises a heavy chain CDR1, a heavy chain CDR2, and a heavy chain CDR3 set forth in SEQ ID NOs: 1-3, respectively, and a light chain CDR1, a light chain CDR2, and a light chain CDR3 set forth in SEQ ID NOs: 4-6, respectively.

In the present disclosure, the term “complementarity determining region” or “CDR” refers to highly variable regions of heavy and light chains of an immunoglobulin as defined by Kabat et al. (Kabat et al., Sequences of proteins of immunological interest, 5th Ed., US Department of Health and Human Services, NIH, 1991, and later editions). There are three heavy chain CDRs and three light chain CDRs. Herein, the terms “CDR” and “CDRs” are used to refer to regions comprising one or more, or even all, of the major amino acid residues contributing to the binding affinity of an antibody for an antigen or epitope it recognizes, depending on the situation. In another specific embodiment, the CDR regions or CDRs refer to highly variable regions of heavy and light chains of an immunoglobulin as defined by IMGT.

In some embodiments, the sc-Fv comprises a heavy chain variable region set forth in any one of SEQ ID NOs: 7-11 (preferably SEQ ID NO: 8) and a light chain variable region set forth in any one of SEQ ID NOs: 12-15 (preferably SEQ ID NO: 13).

In the present disclosure, the term “scFv” refers to a molecule comprising an antibody heavy chain variable domain (or region; VH) and an antibody light chain variable domain (or region; VL) linked by a linker. Such scFv molecules may have a general structure of NH₂—VL-linker peptide (linker)-VH—COOH or NH₂—VH-linker peptide (linker)-VL-COOH.

In the present disclosure, the term “linker peptide” may be a peptide which is flexible or rigid, e.g., consisting of repeated GGGGS amino acid sequences or variants thereof, e.g., 1-4 repeated variants (Holliger et al. (1993), Proc. Natl. Acad. Sci. USA 90: 6444-6448). Other linker peptides that can be used in the present disclosure are described by Alfthan et al., (1995), Protein Eng., 8: 725-731; Choi et al., (2001), Eur J. Immunol., 31: 94-10⁶; Hu et al., (1996), Cancer Res., 56: 3055-3061; Kipriyanov et al., (1999), J. Mol. Biol., 293: 41-56; and Roovers et al., (2001), Cancer Immunol.

In some embodiments, the number of amino acids of the linker peptide is 1-30, which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, preferably 5-20.

In some embodiments, the amino acids of the linker peptide are nonsense polypeptides that do not have additional functions (e.g., protein localization, enzyme cleavage sites, etc.) other than linking.

In some embodiments, the linker peptide is a flexible linker peptide.

In some embodiments, the amino acid sequence of the linking peptide is selected from one or more of Gly, Ser, Pro, Ala, and Glu.

In some embodiments, the amino acid sequence of the linker peptide is selected from (GGGGS)n, (GGGS)n, (GGS)n, (GS)n, or (G)n, wherein n is selected from 1, 2, 3, 4, 5, or 6.

Modified forms of single-chain antibodies are also within the protection scope of the present disclosure, e.g., those modified by covalent attachment of polyethylene glycol or other suitable polymers. Variants of single-chain antibodies are also within the scope of the present disclosure, wherein variants of the heavy chain CDR1-CDR3 and the light chain CDR1-CDR3 may each comprise up to 3 amino acid mutations (e.g., 1, 2, or 3 amino acid replacements, deletions, or additions, or any combination thereof) as compared to any one of combinations of complementarity determining regions set forth in SEQ ID NOs: 1-6; preferably, the mutations are conservative mutations. “Conservative replacement” refers to a replacement of amino acids in a protein with other amino acids having similar characteristics (e.g., charge, side-chain size, hydrophobicity/hydrophilicity, backbone conformation and rigidity, etc.), such that changes can be frequently made without changing the biological activity of the protein.

The replacements which are generally regarded as conservative replacements are replacements for one another in aliphatic amino acids Ala, Val, Leu, and Ile, an exchange of hydroxyl residues Ser and Thr, an exchange of acidic residues Asp and Glu, a replacement between amide residues Asn and Gln, an exchange of basic residues Lys and Arg, and a replacement between aromatic residues Phe and Tyr. Those skilled in the art know that, generally, a single amino acid replacement in a non-essential region of a polypeptide does not substantially change the biological activity (see, e.g., Watson et al. (1987) Molecular Biology of the Gene, The Benjamin/Cummings Pub. Co., p224, (4^{th} ed.)). In addition, the replacement of amino acids with similar structure or function is unlikely to disrupt the biological activity.

Variants of SEQ ID NOs: 7-15 are also within the protection scope of the present disclosure, and the variants may have 90%-99.9% identity, e.g., 90%, 91%, 92%, 93%, 94%, 95%, 96%, 97%, 98%, or 99% identity to amino acid sequences set forth in SEQ ID NOs: 7-15. The variants may also have the modifications or conservative replacements as described above.

In a particular embodiment, an EpCAM-targeted chimeric antigen receptor (abbreviated as EpCAM-CAR) comprises an antigen-binding region, a CD28 hinge region, a CD28 transmembrane region, and an intracellular signaling domain.

The antigen-binding region is a humanized scFv antibody that binds to an EpCAM antigen.

The VH sequence of the humanized antibody is:

QVQLVQSGAEVKKPGASVKVSCKASGYTFTNYWINWVRQAPGQGLEWIG
NIYPSYIYTNYNQEFKDKVTLTVDESTSTAYMELSSLRSEDTAVYYCTR
SPYGYDEYGLDYWGQGTTVTVSS.

The sequence of the G4S×3 linker region is:

GGGGSGGGGSGGGGS.

The VL sequence of the humanized antibody is:

DIQLTQSPSSLSASVGDRVTMTCKSSQSLLNTRNQKNYLTWYQQKPGKA
PKLLIYWASTRESGVPSRFSGSGSGTDFTLTISSLQPEDFATYYCQNDY
VYPLTFGQGTKLEIK.

The sequence of the CD28 hinge region is:

IEVMYPPPYLDNEKSNGTIIHVKGKHLCPSPLFPGPSKP.

The sequence of the CD28 transmembrane region is:

FWVLVVVGGVLACYSLLVTVAFIIFWV.

The sequence of the CD28 co-stimulatory signaling region is:

RSKRSRLLHSDYMNMTPRRPGPTRKHYQPYAPPRDFAAYRS.

The sequence of the CD3(co-stimulatory signaling region is:

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

Specifically, the amino acid sequence of EpCAM-CAR is set forth in SEQ ID NO: 16.

A second aspect of the present disclosure relates to a method for preventing or treating a tumor in a patient in need thereof, which comprises administering to the patient a therapeutically effective amount of the immune cell as described above or a pharmaceutical composition.

The tumor as defined in the first aspect of the present disclosure is also suitable for use in the second aspect.

It should be understood that the envisaged treatment method may further comprise administering other therapeutic entities, particularly preferably immunotherapeutic entities, including viral cancer vaccines (e.g., adenovirus vectors encoding cancer-specific antigens), bacterial cancer vaccines (e.g., non-pyrogenic Escherichia coli expressing one or more cancer-specific antigens), yeast cancer vaccines, N-803 (also referred to as ALT-803, ALTOR), chemotherapeutics, antibodies (e.g., those binding to tumor-associated antigens or patient-specific tumor neoantigens), stem cell grafts (e.g., allogeneic or autologous), and tumor-targeted cytokines (e.g., NHS-IL12 or IL-12 conjugated with tumor-targeted antibodies or fragments thereof). In some embodiments, the envisaged treatment method further comprises administering a radiation therapy to the patient. In some embodiments, the envisaged treatment method further comprises performing surgery, e.g., tumorectomy, on the patient.

The mode of administration may be intravenous administration and/or intracorporeal administration (preferably intraperitoneal administration or intravesical administration).

The “patient” is a mammal, including, but not limited to, humans, monkeys, pigs, and other farm animals, sport animals, pets, primates, horses, dogs, cats, pandas, rodents (including mice, rats, or guinea pigs), etc.

The embodiments of the present disclosure will be described in detail below with reference to the examples. It should be understood that these examples are for illustrative purposes only and are not intended to limit the scope of the present disclosure. Procedures without specified conditions in the following examples are preferably conducted with reference to the guidelines provided in the present disclosure, in accordance with experimental manuals or conventional conditions in the art, with reference to other experimental procedures known in the art, or in accordance with conditions recommended by the manufacturer.

In the specific examples below, the measurements for starting material components may have slight errors within the range of weighing accuracy unless otherwise specified. For the temperature and time parameters, acceptable errors from instrument test accuracy or operating accuracy are permitted.

For the preparation and effect characterization of the EpCAM humanized antibody, the construction of CAR-T cells targeting EpCAM, and other related contents, see the Chinese Patent Application with Publication No. CN113527491A and Publication Date of Oct. 22, 2021. All documents mentioned in the present disclosure are incorporated herein by reference, just as each document is cited separately as a reference. The cited documents involved in the present application are incorporated by reference in their entireties for all purposes unless otherwise in conflict with the objective and/or technical solutions of the present application. Where the cited documents are involved in the present disclosure, definitions of relevant technical features, terms, nouns, phrases, etc., in the cited documents, are also cited. Where the cited documents are involved in the present disclosure, the examples and preferred embodiments of the related art cited are also incorporated herein by reference, but to the extent that it is possible to implement the present disclosure. It should be understood that where the cited content conflicts with the description of the present application, the present application shall prevail or amendments shall be made according to the description of the present application.

### Example 1. Clinical Study of EpCAM-Targeted Chimeric Antigen Receptor-Modified Autologous T Cells for Advanced Gastric Cancer

### 1.1. Study Drug

EpCAM-targeted chimeric antigen receptor-modified autologous T cells with the code name of EpCAM-CAR-T. EpCAM-CAR-T contains an antigen-recognition domain, comprising a heavy chain variable region and a light chain variable region. The sequence of the heavy chain variable region is set forth in SEQ ID NO: 8, and the sequence of the light chain variable region is set forth in SEQ ID NO: 13; the amino acid sequence of the chimeric antigen receptor of EpCAM-CAR-T is set forth in SEQ ID NO: 16.

### 1.2 Mode of Administration

### Intravenous Administration

### 1.3 Design of Trial

This is a multicenter, single-arm, open-label, dose-escalation clinical study. This is an open-label clinical study for evaluating the safety and efficacy of EpCAM-CAR-T in the treatment of advanced gastric cancer. An initial dose of 3×10⁵ cells/kg was recommended, and dose escalation was performed according to the 3+3 dose escalation principle.

### 1.4 Results

Six patients with advanced gastric cancer were enrolled in this study, and histopathologic staining showed that the expression rate of EpCAM was 10%-90%. After treatment with 3×10⁵ cells/kg or 1×10⁶ cells/kg EpCAM-CAR-T, good safety was shown in the patients, with no dose-limiting toxic reactions occurring in any of the patients. The efficacy evaluation showed that disease control occurred in 5 patients, with a disease control rate (DCR) of 83.3%; of these patients, 2 exhibited a partial response (PR).

| Patient | Clinical diagnosis | EpCAM expression | Results of treatment |
|---|---|---|---|
| 1 | Extensive abdominal | Expression | After the administration of 3 × 10⁵ cells/kg |
| | cavity metastases | rate: 0.9, and | EpCAM-CAR-T, the safety evaluation on day |
| | of gastric corpus | H-score: 2.9 | 28 showed that there were no dose-limiting |
| | cancer and ascites | | toxic reactions. The efficacy evaluation |
| | | | showed disease control. |
| 2 | Gastric antrum | Expression | After the administration of 3 × 10⁵ cells/kg |
| | cancer involving | rate: 0.3, and | EpCAM-CAR-T, the safety evaluation on day |
| | the duodenum and | H-score: 1.4 | 28 showed that there were no dose-limiting |
| | head of pancreas, | | toxic reactions. The efficacy evaluation on day |
| | and ascites | | 28 showed disease control. The efficacy |
| | | | evaluations on week 32 and week 40 showed a |
| | | | partial response. |
| 3 | Gastric corpus | Expression | After the administration of 3 × 10⁵ cells/kg |
| | malignant tumor | rate: 0.1, and | EpCAM-CAR-T, the safety evaluation on day |
| | with extensive | H-score: 1.05 | 28 showed that there were no dose-limiting |
| | abdominal cavity | | toxic reactions. The efficacy evaluation |
| | metastases, ovarian | | showed disease control. |
| | metastases, and | | |
| | bone metastases, | | |
| | and ascites | | |
| 4 | Gastric cancer | Expression | After the administration of 1 × 10⁶ cells/kg |
| | with abdominal | rate: 0.1, and | EpCAM-CAR-T, the safety evaluation on day |
| | cavity lymph | H-score: 1.1 | 28 showed that there were no dose-limiting |
| | node metastases | | toxic reactions. The efficacy evaluation on day |
| | | | 28 showed disease control, and the efficacy |
| | | | evaluations on week 16 and week 24 showed a |
| | | | partial response. |
| 5 | Multiple metastatic | Expression | After the administration of 1 × 10⁶ cells/kg |
| | tumors in the | rate: 0.9, and | EpCAM-CAR-T, the safety evaluation on day |
| | liver and abdominal | H-score: 2.45 | 28 showed that there were no dose-limiting |
| | cavity after | | toxic reactions. The efficacy evaluation on |
| | radical surgery | | week 8 showed disease progression. |
| | for gastric cancer | | |
| 6 | Multiple metastases | Expression | After the administration of 1 × 10⁶ cells/kg |
| | to both lungs after | rate: 0.9, and | EpCAM-CAR-T, the safety evaluation on day |
| | radical surgery | H-score: 2.15 | 28 showed that there were no dose-limiting |
| | for gastric cancer | | toxic reactions. The efficacy evaluation on |
| | | | week 8 showed disease control. |

Patient 2 in the 3×10⁵ cells/kg dose group, male, 40 years old, with gastric antrum cancer involving the duodenum and head of pancreas, pathologically diagnosed as poorly differentiated adenocarcinoma (of the antrum), partly signet-ring cell carcinoma, underwent laparoscopic exploration and gastrojejunostomy in January 2021, and failed multiple lines of treatment including a tegafur monotherapy, chemotherapies with a 7-cycle FTP protocol and a FOLFIRI protocol, apatinib mesylate tablets, etc. The patient received treatment by an infusion with 3×10⁵/kg CAR-T cells on Nov. 16, 2021, and no CRS or ICANS occurred after the reinfusion. The results of the efficacy evaluation of the patient are shown in the table below. The patient was still alive 60 weeks after the administration.

| | Baseline | Week 4 | Week 8 | Week 32 | Week 40 |
|---|---|---|---|---|---|
| Gastric corpus | 45.6 mm | 40.73 mm | 41.39 mm | 30.75 mm | 28.84 mm |
| tumor mass | | | | | |
| Lymph node | Present | Volume | Volume | Volume | Volume |
| group 8 | | reduced | reduced | reduced | reduced |
| Ascites | Large | Significantly | Disappeared | Disappeared | Disappeared |
| | amount | reduced | | | |
| Pelvic effusion | Large | Significantly | Significantly | Significantly | Significantly |
| | amount | reduced | reduced | reduced | reduced |
| New lesion | None | None | None | None | None |
| CEA (0-5 ng/ml) | 7.23 | 9.39 | 7.2 | 3.47 | |
| CA125 (0-35 U/ml) | 67.4 | 31.5 | 22.8 | 17.6 | |

Patient 1 in the 1×10⁶ cells/kg dose group, male, 50 years old, gastric corpus adenocarcinoma, underwent left-sided gastric arteriography and arterial perfusion chemoembolization (raltitrexed protocol) in May 2021, with no indication for radical surgery. MR enhancement on Feb. 15, 2022 showed gastric cancer with gastric omental bursa and lymph node metastases adjacent to the gastric antrum with multiple intrahepatic nodes, which was considered to be disease progression in the patient after the second-line treatment. The patient received treatment with 1×10⁶ cells/kg CAR-T cells on Jun. 8, 2021. The results of the efficacy evaluation of the patient are shown in the table below. The patient recovered to the point of having an indication for radical surgery 24 weeks after the administration as judged by the investigator, and underwent radical surgery for gastric cancer on Jan. 3, 2023, and the patient was still alive 52 weeks after the administration.

| | Baseline | Week 4 | Week 8 | Week 16 | Week 24 |
|---|---|---|---|---|---|
| Lymph node | 22.91 mm | 20.32 mm | 16.0 mm | 13.90 mm | 12.21 mm |
| group 8 | | | | | |
| Lymph node | 15.01 mm | 12.76 mm | 11.99 mm | 11.68 mm | 11.80 mm |
| group 9 | | | | | |
| New lesion | None | None | None | None | None |

### Example 2. Clinical Study of EpCAM-Targeted Chimeric Antigen Receptor-Modified Autologous T Cells for Advanced Digestive System Malignant Tumor

### 2.1 Study Drug

EpCAM-targeted chimeric antigen receptor-modified autologous T cells with the code name of EpCAM-CAR-T. EpCAM-CAR-T contains an antigen-recognition domain, including a heavy chain variable region and a light chain variable region. The sequence of the heavy chain variable region is set forth in SEQ ID NO: 8, and the sequence of the light chain variable region is set forth in SEQ ID NO: 13; the amino acid sequence of the chimeric antigen receptor of EpCAM-CAR-T is set forth in SEQ ID NO: 16.

### 2.2 Mode of Administration

### Intravenous Administration

### 2.3 Design of Trial

This is a multicenter, single-arm, open-label, dose-escalation clinical study. This is an open-label clinical study for evaluating the safety and efficacy of EpCAM-CAR-T in the treatment of an advanced gastrointestinal tumor. An initial dose of 3×10⁵ cells/kg was recommended, and dose escalation was performed according to the 3+3 dose escalation principle.

### 2.4 Results

Three patients with advanced gastrointestinal malignant tumors were enrolled, all with colorectal cancer with multiple metastases. Histopathological staining showed that the expression rate of EpCAM was 80%-90%. 28 days after the administration of 3×10⁵ cells/kg EpCAM-CAR-T, good safety was shown in three patients, with no dose-limiting toxic reactions occurring in any of the patients. The efficacy evaluation showed that disease control occurred in 2 of the 3 patients, with a disease control rate (DCR) of 66.7%.

| Patient | Clinical diagnosis | EpCAM expression | Results of treatment |
|---|---|---|---|
| 1 | After surgery for colon | Expression | After the administration of 3 × 10⁵ cells/kg |
| | cancer and left hepatic | rate: 0.8, and | EpCAM-CAR-T, the safety evaluation on day |
| | metastatic tumor, multiple | H-score: 2.6 | 28 showed that there were no dose-limiting |
| | metastases in the liver | | toxic reactions. The efficacy evaluation |
| | and to both lungs | | showed disease progression. |
| 2 | After surgery for right- | Expression | After the administration of 3 × 10⁵ cells/kg |
| | sided colon cancer, multiple | rate: 0.8, and | EpCAM-CAR-T, the safety evaluation on day |
| | metastases to both lungs, | H-score: 1.2 | 28 showed that there were no dose-limiting |
| | and rectal, pelvic and | | toxic reactions. The efficacy evaluation |
| | retroperitoneal lymph | | showed disease control. |
| | node metastases | | |
| 3 | After surgery for the | Expression | After the administration of 3 × 10⁵ cells/kg |
| | hepatic metastasis of | rate: 0.9, and | EpCAM-CAR-T, the safety evaluation on day |
| | sigmoid colon cancer, | H-score: 2.0 | 28 showed that there were no dose-limiting |
| | multiple metastases in the | | toxic reactions. The efficacy evaluation |
| | liver and to both lungs | | showed disease control. |

### Example 3. Blocking Effect of EpCAM-Targeted Chimeric Antigen Receptor-Modified Autologous T Cells on Metastatic Tumor In Vivo

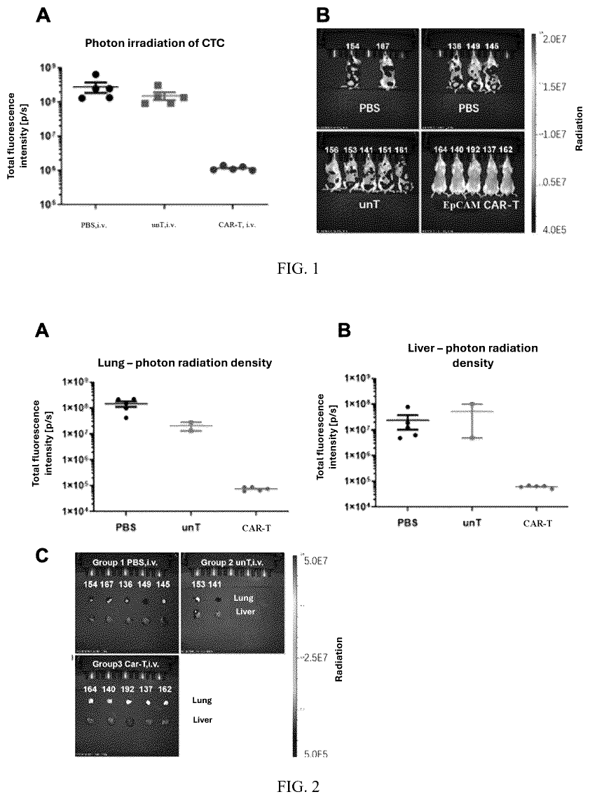

To demonstrate the blocking effect of EpCAM-CAR-T on a metastatic tumor formed by CTCs (the amino acid sequence of the chimeric antigen receptor of EpCAM-CAR-T is set forth in SEQ TD NO: 16), we constructed a pharmacodynamic model that simulates CAR-T in treating a metastatic tumor formed by CTCs. M-NSG mice were first inoculated subcutaneously with HCT116 cells without fluorescent labeling. EpCAM-CAR-T cells, unT, or DPBS were reinfused via the tail vein after tumors grew to 100-150 mm³. Two days after CAR-T reinfusion, mice were inoculated with CTC cells (simulated by HCT116-Luc) via the tail vein to verify whether EpCAM-CAR-T cells could clear CTCs and block them from forming metastases in vivo. The results are shown in FIGS. 1A and 1B. The results showed that there was no metastasis fluorescence in the CAR-T treatment group, while multiple metastases were formed in mice of the DPBS and unT groups, and the fluorescence intensity was significantly higher than that of the treatment group. To determine whether CTCs form metastases in key organs and the therapeutic effect of CAR-T, ex vivo imaging analysis of the lungs and liver was performed at the end of the study. The results showed that there were strong fluorescence signals in the liver and lungs of both the unT group and the DPBS group, while there were no significant fluorescence signals in the lungs and liver of the EpCAM-CAR-T treatment group. The results are shown in FIG. 2. The above results suggest that EpCAM-CAR-T cells can clear CTCs and prevent them from forming metastatic lesions in key organs.

### Example 4. Study of EpCAM-Targeted Chimeric Antigen Receptor-Modified Autologous T Cells in Treatment of Abdominal Metastatic Tumor of Gastric Cancer

To study whether EpCAM-CAR-T (the amino acid sequence of the chimeric antigen receptor of EpCAM-CAR-T is set forth in SEQ ID NO: 16) can clear abdominal metastatic tumors of gastric cancer and whether the chemotherapeutic drug docetaxel can enhance the efficacy of CAR-T, we constructed an abdominal cavity metastasis model of gastric cancer based on a gastric cancer cell line MKN-45-luc. Modeling: human gastric cancer cells MKN-45-luc were cultured in an RPMI-1640 culture medium with 10% FBS in an incubator at 37° C. with 5% CO₂. Before continuous cell culturing for ten generations, the cells were inoculated into the abdominal cavity of mice, about 1.5×10⁶ MKN-45-luc cells were resuspended in PBS, and inoculated into NCG mice by intraperitoneal injection in a volume of 100 L. Grouping and administration: 7 days after the inoculation, the growth of tumor cells in the abdominal cavity was detected using a small animal in vivo imaging fluorometer. The mice were randomly grouped according to the body weight and tumor size (fluorescence intensity), with 5 animals in each group, for a total of 6 groups. The day of grouping was defined as day 0. The grouping and administration regimens are shown in the table below:

| | | | Dose (CAR-T | | Route of | Administration | | |
|---|---|---|---|---|---|---|---|---|
| Group | N | Test sample | cells/mouse) | | administration | volume | | Regimen |
| 1 | 5 | unT | 2M | | i.v. | 200 | μL/mouse | Day 2, |
| | | | | | | | | single dose |
| 2 | 5 | EpCAM- | 0.5M | | i.v. | 200 | μL/mouse | Day 2, |
| | | CAR-T | | | | | | single dose |
| 3 | 5 | EpCAM- | 2M | | i.v. | 200 | μL/mouse | Day 2, |
| | | CAR-T | | | | | | single dose |
| 4 | 5 | Docetaxel | 8 | mg/kg | i.p. | 10 | uL/g | Day 0, |
| | | | | | | | | single dose |
| 5 | 5 | Docetaxel | 1.5 | mg/kg | i.p. | 10 | uL/g | Day 0, |
| | | | | | | | | single dose |

| | | | | | | |
|---|---|---|---|---|---|---|
| 6 | 5 | EpCAM- | 0.5M + 1.5 mg/kg | i.v. + i.p. | 200 μL/mouse + | Day 2 + |

| | | |
|---|---|---|
| CAR-T + | 10 μL/g | Day 0, |
| Docetaxel | | single dose |

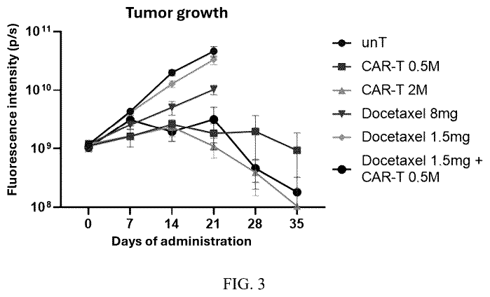

The results of the study are shown in FIG. 3. Compared with control T cells (unT), the 0.5 M and 2 M EpCAM-CAR-T cell treatment groups could effectively inhibit the tumor growth, with a significantly reduced tumor fluorescence value, and had efficacy positively correlated with the dosage. Especially, the 2 M dosage group had a particularly significant tumor inhibitory effect. A single dose of 1.5 mg/kg docetaxel did not produce significant efficacy. Meanwhile, it was observed that the combination of 1.5 mg/kg of docetaxel and 0.5 M EpCAM-CAR-T cells could enhance the tumor inhibitory effect of CAR-T, with a tumor fluorescence value lower than that of the 0.5 M EpCAM-CAR-T treatment group alone. The above results suggest that EpCAM-CAR-T can effectively inhibit the growth of abdominal cavity metastasis tumors induced by gastric cancer cells MKN45-Luc, and the efficacy of EpCAM-CAR-T, when in combination with docetaxel, can be improved.

The above examples only illustrate several embodiments of the present disclosure for the purpose of specific and detailed description, but should not be construed as limiting the scope of the present disclosure. It should be noted that various modifications and improvements can be made by those of ordinary skill in the art without departing from the spirit of the present disclosure, and such modifications and improvements shall fall within the protection scope of the present disclosure. Therefore, the protection scope of the patent of the present disclosure shall be subject to the appended claims, and the description and the drawings can be used to illustrate the content of the claims.

1.-11. (canceled)

12. A method for preventing or treating a tumor in a patient in need thereof, comprising administering to the patient a therapeutically effective amount of immune cells wherein the immune cell is a cell for an adoptive immune cell therapy, which can target and kill an EpCAM-expressing cell, and wherein the tumor expresses EpCAm and is a tumor with a metastasis.

13. The method according to claim 12, wherein the tumor is a gastrointestinal tumor, pancreatic cancer, ovarian cancer, or bladder cancer.

14. The method according to claim 13, wherein the gastrointestinal tumor is gastric cancer or colorectal cancer.

15. The method according to claim 14, wherein the tumor is gastric cancer with a peritoneal metastasis.

16. The method according to claim 14, wherein the tumor is colorectal cancer with a liver metastasis.

17. The method according to claim 12, wherein the immune cell is a T cell, an NK cell, or a DC cell.

18. The method according to claim 12, wherein the cell expresses a chimeric antigen receptor having an extracellular antigen-recognition domain for recognizing an EpCAM antigen.

19. The method according to claim 18, wherein the antigen-recognition domain comprises a sc-Fv, preferably comprising a heavy chain CDR1, a heavy chain CDR2, and a heavy chain CDR3 set forth in SEQ ID NOs: 1-3, respectively, and a light chain CDR1, a light chain CDR2, and a light chain CDR3 set forth in SEQ ID NOs: 4-6, respectively.

20. The method according to claim 19, wherein the sc-Fv comprises a heavy chain variable region set forth in any one of SEQ ID NOs: 7-11 and a light chain variable region set forth in any one of SEQ ID NOs: 12-15.

21. The method according to claim 20, wherein the sc-Fv comprises a heavy chain variable region set forth in SEQ ID NO: 8 and a light chain variable region set forth in SEQ ID NO: 13.

22. The method according to claim 20, wherein the chimeric antigen receptor has an amino acid sequence set forth in SEQ ID NO: 16.

23. A pharmaceutical composition for preventing or treating a tumor, comprising a therapeutically effective amount of immune cells, wherein the immune cell is a cell for an adoptive immune cell therapy, which can target and kill an EpCAM-expressing cell, and wherein the tumor expresses EpCAM and is a tumor with a metastasis.


SEQUENCE LISTING (16 sequences)

SEQ ID NO: 1 - protein, 5 aa, synthetic construct
NYWIN

SEQ ID NO: 2 - protein, 17 aa, synthetic construct
NIYPSYIYTNYNQEFKD

SEQ ID NO: 3 - protein, 12 aa, synthetic construct
SPYGYDEYGLDY

SEQ ID NO: 4 - protein, 17 aa, synthetic construct
KSSQSLLNSRNQKNYLT

SEQ ID NO: 5 - protein, 7 aa, synthetic construct
WASTRES

SEQ ID NO: 6 - protein, 9 aa, synthetic construct
QNDYVYPLT

SEQ ID NO: 7 - protein, 121 aa, synthetic construct
QVQLVQSGAEVKKPGASVKLSCKASGYTFTNYWINWVKQAPGQGLEWIGNIYPSYIYTNYNQEFKDKVTLTVDESTSTAYMELSSLRSEDTAVYYCTRSPYGYDEYGLDYWGQGTTVTVSS

SEQ ID NO: 8 - protein, 121 aa, synthetic construct
QVQLVQSGAEVKKPGASVKVSCKASGYTFTNYWINWVRQAPGQGLEWIGNIYPSYIYTNYNQEFKDKVTLTVDESTSTAYMELSSLRSEDTAVYYCTRSPYGYDEYGLDYWGQGTTVTVSS

SEQ ID NO: 9 - protein, 121 aa, synthetic construct
QVQLVQSGAEVKKPGASVKVSCKASGYTFTNYWINWVRQAPGQGLEWIGNIYPSYIYTNYNQEFKDRVTLTRDTSISTAYMELSRLRSDDTAVYYCTRSPYGYDEYGLDYWGQGTTVTVSS

SEQ ID NO: 10 - protein, 121 aa, synthetic construct
QVQLVQSGAEVKKPGASVKVSCKASGYTFTNYWINWVRQAPGQGLEWIGNIYPSYIYTNYAQKFQGRVTLTRDTSTSTVYMELSSLRSEDTAVYYCTRSPYGYDEYGLDYWGQGTTVTVSS

SEQ ID NO: 11 - protein, 121 aa, synthetic construct
QVQLVQSGAEVKKPGASVKVSCKASGYTFTNYWINWVRQAPGQGLEWIGNIYPSYIYTNYNQEFKDRVTMTRDTSISTAYMELSRLRSDDTAVYYCTRSPYGYDEYGLDYWGQGTTVTVSS

SEQ ID NO: 12 - protein, 113 aa, synthetic construct
DIQLTQSPSSLSASVGDRVTMTCKSSQSLLNSRNQKNYLTWYQQKPGKAPKLLIYWASTRESGVPSRFSGSGSGTDFTLTISSVQPEDFATYYCQNDYVYPLTFGQGTKLEIK

SEQ ID NO: 13 - protein, 113 aa, synthetic construct
DIQLTQSPSSLSASVGDRVTMTCKSSQSLLNTRNQKNYLTWYQQKPGKAPKLLIYWASTRESGVPSRFSGSGSGTDFTLTISSLQPEDFATYYCQNDYVYPLTFGQGTKLEIK

SEQ ID NO: 14 - protein, 113 aa, synthetic construct
DIVLTQSPDSLAVSLGERATMNCKSSQSLLNSRNQKNYLTWYQQKPGQPPKLLIYWASTRESGVPDRFSGSGSGTDFTLTISSLQAEDVAVYYCQNDYVYPLTFGQGTKLEIK

SEQ ID NO: 15 - protein, 113 aa, synthetic construct
EIVLTQSPATLSLSPGERATMSCKSSQSLLNSRNQKNYLTWYQQKPGQAPRLLIYWASTRESGIPARFSGSGSGTDFTLTISSLEPEDFAVYYCQNDYVYPLTFGQGTKLEIK

SEQ ID NO: 16 - protein, 468 aa, synthetic construct
QVQLVQSGAEVKKPGASVKVSCKASGYTFTNYWINWVRQAPGQGLEWIGNIYPSYIYTNYNQEFKDKVTLTVDESTSTAYMELSSLRSEDTAVYYCTRSPYGYDEYGLDYWGQGTTVTVSSGGGGSGGGGSGGGGSDIQLTQSPSSLSASVGDRVTMTCKSSQSLLNTRNQKNYLTWYQQKPGKAPKLLIYWASTRESGVPSRFSGSGSGTDFTLTISSLQPEDFATYYCQNDYVYPLTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR